(19) United States
(12) Patent Application Publication
(10) Pub. No.: US 2001/0034029 A1
(43) Pub. Date: Oct. 25, 2001

(54) Method of detecting mutation in base sequence of nucleic acid

(75) Inventor: Hideshi Fujiwake, Kyoto (JP)

Correspondence Address: ARENT FOX KINTNER PLOTKIN & KAHN, PLLC, 1050 Connecticut Avenue, N.W., Suite 600, Washington, DC, 20036-5339, US

(73) Assignee: SHIMADZU CORPORATION

(21) Appl. No.: 09/828,211

(22) Filed: Apr. 9, 2001

(30) Foreign Application Priority Data
Apr. 19, 2000 (JP) 2000-118587

(51) Int. Cl. C12Q 1/68

(52) U.S. Cl. 435/6

(57) ABSTRACT

Normal DNA (15) and mutational DNA (17) are mixed and subjected to PCR amplification (A). Fluorescent oligonucleotides (19a, 19b, 19c) complementary to any of exons (15a, 15b, 15c) and labeled with fluorescent materials (F1, F2, F3) having different fluorescence spectral characteristics are prepared (B) and hybridized with the amplified substance for forming homoduplexes (21a, 21b, 21c, 23b) and heteroduplexes (23a, 23c) (C). Since the heteroduplexes (23a, 23c) have a lower melting temperature than the homoduplexes (21a, 21b, 21c, 23b), analysis is made with an ion pair chromatograph having a reversed phase column set at the melting temperature for discriminating and detecting relatively quickly eluting fluorescent oligonucleotides (19a, 19c) thereby detecting mutational exons.

### BACKGROUND OF THE INVENTION

1. Field of the Invention

The present invention relates to a method of detecting mutation in the base sequence of nucleic acid including DNA (deoxyribonucleic acid) and RNA (ribonucleic acid).

2. Description of the Prior Art

It has been clarified that many cancers and genetic diseases are caused by mutation in the base sequence of DNA. The mutation in the base sequence is generally monobasic substitution. A number of methods have been proposed in the technical field of detecting such mutation in the base sequence. Some of the methods are now illustrated.

1) DNA (RNA) Sequence:

The base sequence of a substance to be analyzed is directly analyzed and determined. Although this method is most reliable, it's disadvantage is the high cost required for a series of operations. Further, a large-scale automation line is necessary for improving the throughput.

2) DNA Chip:

A number of oligonucleotides are fixed onto a glass surface and selectively hybridized with a substance to be analyzed such as a DNA fragment for thereafter detecting a signal based on the hybridization, generally a fluorescent signal, and comparing the same with a normal one thereby estimating presence/absence of mutation in the sequence of the substance.

However, a DNA chip itself is extremely high-priced and the number of oligonucleotides fixed onto the chip must be varied with the substance, disadvantageously leading to a high cost.

3) SSCP (single strand conformation polymorphism) Method:

Double stranded DNA (RNA) employed as a sample is converted to single stranded DNA for thereafter electrophoretically detecting the difference of stereochemical structure of the single stranded DNA which varies with the base sequence, thereby estimating presence/absence of mutation in the base sequence.

However, in this method, electrophoretic conditions must be studied every sample, and it is disadvantageously difficult to improve the throughput due to employment of gel electrophoresis.

4) DHPLC (denaturing high performance liquid chromatography) Method:

The DHPLC method which utilizes ion pair chromatography is disclosed in, for example, U.S. Pat. No. 5795976. This method is now described with reference to FIGS. 1(A) and 1(B).

(A) DNA fragments are subjected to PCR (polymerase chain reaction) amplification. It is assumed that normal DNA 2 having normal base sequence and mutational DNA 4 having mutational base sequence are mixed with each other as the DNA fragments (see FIG. 1(A)). The base sequence of the mutational DNA 4 is different from that of the normal DNA 2 in underlined portions.

(B) The normal DNA 2 and the mutational DNA 4 mixed with each other are thermally denatured into single stranded DNA, and thereafter the temperature is reduced for re-bonding the same. Consequently, homoduplexes 2a and 4a are formed by re-bonding source pair single strands while heteroduplexes 2b and 4b are also formed by bonding single strands different from the source pair single strands (see FIG. 1(B)).

The homoduplexes 2a and 4a, which are identical in base sequence to the normal DNA 2 and the mutational DNA 4 respectively, form hydrogen bonds as to all base pairs. However, the heteroduplexes 2b and 4b have portions where corresponding bases are inappropriate, i.e., mismatching portions (underlined portions in FIG. 1(B)) forming no hydrogen bonds. Therefore, the homoduplexes 2a and 4a and the heteroduplexes 2b and 4b are different in stability from each other, and the melting temperature, at which 50% of the total concentration of the double-stranded DNA is denatured to single stranded DNA, of the heteroduplexes 2b and 4b is reduced as compared with that of the homoduplexes 2a and 4a.

(C) The homoduplexes 2a and 4a and the heteroduplexes 2b and 4b are separated from each other by employing the principle of ion pair chromatography and setting a reversed phase column at the melting temperature of the heteroduplexes 2b and 4b. The DNA fragments which formed the heteroduplexes 2b and 4b cleave into single strands, and are detected faster than double strands. Therefore, when two detection peaks appear, it follows that the homoduplexes 2a and 4a and the heteroduplexes 2b and 4b are present in the PCR product, and hence it is understood that mutational ones have been present in the inspected sites of the DNA fragments before PCR amplification.

In the DHPLC method, mutation in the base sequence is inspected in units of exons. The exon is a part of the base sequence of DNA, ultimately forming information of protein biosynthesis as amino acid sequence, to be read and translated.

Assuming that the DHPLC method is employed for simultaneously analyzing mutation of a plurality of exons (inspected sites), it is impossible to investigate the inspected site(s) having mutational base sequence. Therefore, in the DHPLC method, only mutation in the base sequence of one inspected site can be determined by single analysis. Therefore, in order to inspect mutation in a plurality of types of inspected sites, a series of operations of heating, re-bonding and analysis must be performed for each inspected site, disadvantageously leading to increase of the time and the cost required for the analysis.

### SUMMARY OF THE INVENTION

Accordingly, an object of the present invention is to provide a method of detecting mutation in the base sequence of nucleic acid capable of discriminating and inspecting mutation in the base sequence of a plurality of types of inspected sites by performing a series of operations of re-bonding and analysis only once.

According to the present invention, a method of detecting mutation in the base sequence of nucleic acid includes the following steps (A) and (B):

(A) a bonding step of hybridizing an object of analysis consisting of nucleic acid or a nucleic acid fragment including a plurality of inspected sites to be subjected to inspection of mutation in the base sequence with a plurality of types of oligonucleotides having base sequence complementary to any of the inspected sites having normal base sequence and labeled to be discriminable from each other for forming duplexes, and

(B) a detection step of employing an ion pair chromatograph comprising a reversed phase column serving as a separation column and a detector capable of discriminating and detecting the labeled oligonucleotides and setting the separation column at a temperature causing difference in stability between hetero- and homoduplexes included in the duplexes for analyzing the object of analysis.

In more detail, first, an object of analysis including a plurality of inspected sites is prepared. If the quantity of the object of analysis is small, it is preferable to amplify the object of analysis. An exemplary preferable amplification step is a PCR step. In order to suppress the cost for PCT reaction, the PCR step is preferably carried out only once.

A plurality of oligonucleotides having base sequence complementary to any of a plurality of types of inspected sites having normal base sequence and labeled to be discriminable from each other are prepared. While radioisotopes can be used as labels, preferable labeling materials are fluorescent materials. Oligonucleotides labeled with fluorescent materials are referred to as fluorescent oligonucleotides. The fluorescent oligonucleotides can be readily discriminated from each other by fluorescence spectra specific to the fluorescent materials. Description is made with reference to the fluorescent oligonucleotides.

The fluorescent oligonucleotides are hybridized with corresponding ones of the inspected sites of the object of analysis. In this hybridization, the object of analysis mixed with the fluorescent oligonucleotides is thermally denatured into single stranded DNA, and the temperature is thereafter reduced for bonding the single stranded DNA of the object of analysis with the fluorescent oligonucleotides. At this time, a homoduplex is formed in an inspected site having normal base sequence while a heteroduplex is formed in an inspected site having mutational base sequence.

In the detection step, utilizing an ion pair chromatograph comprising a reversed phase column serving as a separation column and a detector capable of discriminating and detecting labels (fluorescent materials in this example) and setting the separation column at a temperature causing difference in stability between hetero- and homoduplexes, the object of analysis hybridized with the fluorescent oligonucleotides is introduced into the separation column along with a mobile phase mixed with an ion pair reagent. In the column, heteroduplexes are dissociated in a higher ratio than homoduplexes. Since dissociated fluorescent oligonucleotides elute in advance of hybridized fluorescent oligonucleotides, the fluorescent oligonucleotides having formed heteroduplexes elute in advance. The term “temperature causing difference in stability between hetero- and homoduplexes” stands for a temperature at which hetero- and homoduplexes are denatured and dissociated in different ratios, such as the melting temperature of the heteroduplexes or a temperature around the same.

A chromatogram of labels obtained through the detection step is observed for determining an inspected site corresponding to a label having a single peak as non-mutational while determining an inspected site corresponding to a label having two peaks as mutational. Thus, it is possible to recognize an inspected site forming a heteroduplex, and hence presence/absence of mutation in the base sequence can be investigated as to a plurality of inspected sites by single analysis.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIGS. 1(A) and 1(B) are diagrams for illustrating a DHPLC method;

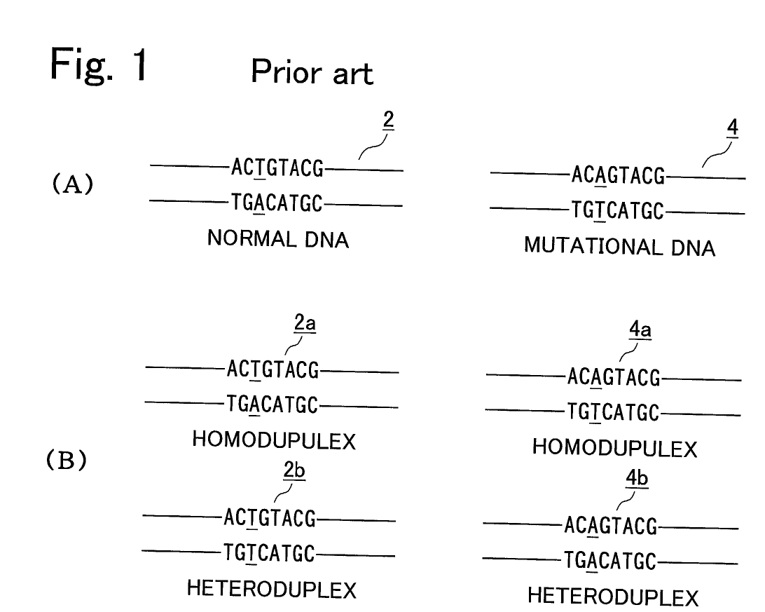

FIG. 2 is a schematic passage structural diagram showing an exemplary ion pair chromatograph;

FIGS. 3(A) to 3(C) are diagrams for illustrating an embodiment of the present invention; and

FIG. 4 is a waveform diagram showing a chromatogram in the embodiment.

### DESCRIPTION OF THE PREFERRED EMBODIMENTS

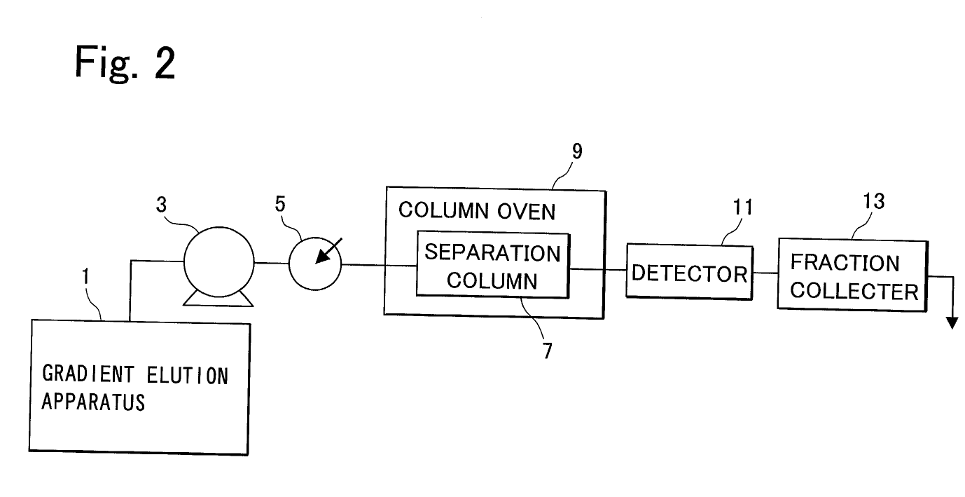

FIG. 2 is a schematic passage structural diagram showing an exemplary ion pair chromatograph employed for the present invention.

A mobile phase is an acetonitrile solution containing triethylamine serving as an ion pair reagent. A gradient elution apparatus 1 supplies the mobile phase while varying the acetonitrile concentration thereof.

The gradient elution apparatus 1 is connected with a feed pump 3 feeding the mobile phase to a separation column 7. The separation column 7 is a reversed phase column. An exemplary reversed phase column has an internal surface formed by a nonporous material such as a nonporous polymer or nonporous silica, which is modified with an alkyl group such as an octadecyl group having 18 carbons connected in a straight-chain manner. Another exemplary reversed phase column is charged with a filler which has a base material of a nonporous material such as a nonporous polymer or nonporous silica bonding an octadecyl group therewith.

A mobile phase passage between the feed pump 3 and the separation column 7 is provided with an injector 5 injecting a sample solution into the mobile phase passage. A column oven 9 adjusting the column temperature is provided around the separation column 7.

An elution side of the separation column 7 is connected to a detector 11 detecting an eluting component. The detector 11 is formed by that capable of discriminating a plurality of fluorescent materials such as three types of fluorescent materials F1, F2 and F3 that have different fluorescence spectral characteristics.

A fraction collector 13 fractioning an eluent on the basis of an output of the detector 11 is provided downstream the detector 11.

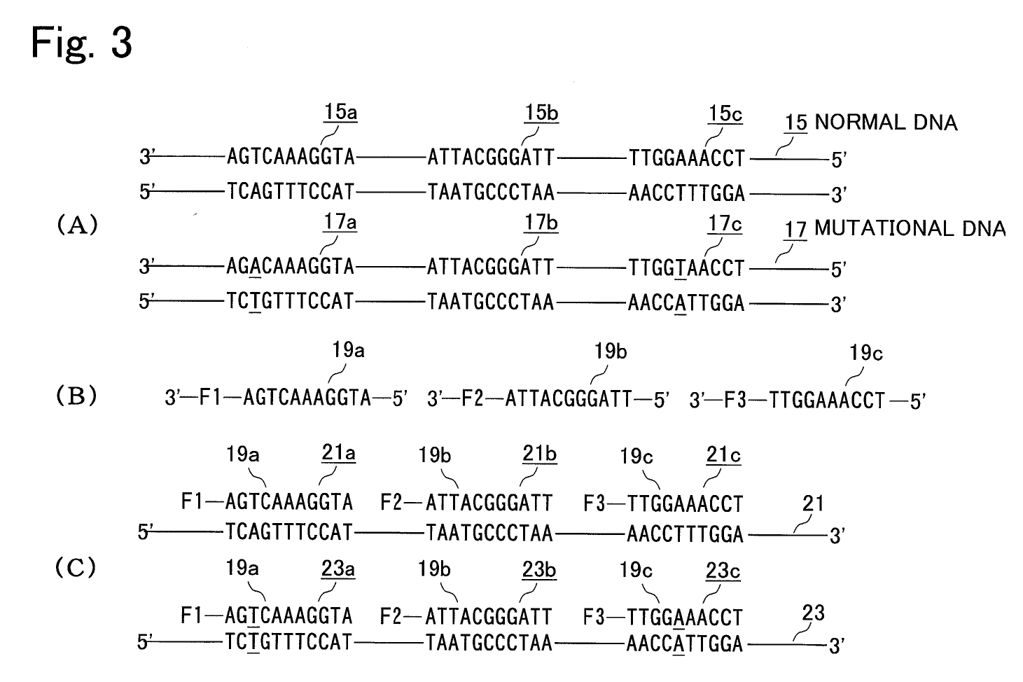

FIGS. 3(A), 3(B) and 3(C) are diagrams for illustrating an embodiment of a method of detecting mutation in the base sequence of nucleic acid according to the present invention. This embodiment shall now be described with reference to FIGS. 2 and 3(A) to 3(C).

A DNA fragment (object of analysis) containing a plurality of exons is subjected to POR amplification. In this example, both normal DNA 15 and mutational DNA 17 are present as objects of analysis (see FIG. 3(A)). Referring to FIGS. 3(A) to 3(C), symbols A, C, G and T denote adenine, cytosine, guanine and thymine respectively. Three exons 15a, 15b and 15c to be inspected are present in the normal DNA 15. Three exons 17a, 17b and 17c to be inspected are present in the mutational DNA 17, and it is assumed that the exons 17a and 17c have mutational base sequence (underlined portions in FIG. 3(A)) as compared with the exons 15a and 15c.

A plurality of types of oligonucleotides (fluorescent oligonucleotides) 19a, 19b and 19c having base sequence complementary to the sequence of first chains forming the exons 15a, 15b and 15c having normal base sequence and labeled with the fluorescent materials F1, F2 and F3 respectively are prepared (see FIG. 3(B)). The oligonucleotides to be prepared may not correspond to all exons, but may correspond to only portions of the exons to be inspected. When inspecting mutation in the base sequence of the same exons as to a number of samples, the cost can be reduced by previously preparing a large quantity of fluorescent oligonucleotides.

The normal DNA 15 and the mutational DNA 17 subjected to PCR amplification and the fluorescent oligonucleotides 19a, 19b and 19c are mixed with each other in a solution, which in turn is heated under a temperature condition of, for example, 95° C. for 10 seconds to thermally denature and dissociate the normal DNA 15 and the mutational DNA 17 and thereafter maintaining the same at a temperature of 60° C. for 30 minutes for preparing a sample solution. Thus, the fluorescent oligonucleotides 19a, 19b and 19c are hybridized with the first chains of the corresponding exons 15a, 15b, 15c, 17a, 17b and 17c respectively, for forming homoduplexes 21a, 21b and 21c in a first chain 21 of the normal DNA 15 while forming heteroduplexes 23a and 23c and a homoduplex 23b in a first chain 23 of the mutational DNA 17 (see FIG. 3(C)). Bases shown with underlines in FIG. 3(C) mismatch in the heteroduplexes 23a and 23c, and hence the heteroduplex 23a has a lower melting temperature than the homoduplex 21a and the heteroduplex 23c has a lower melting temperature than the homoduplex 21c.

The fluorescent oligonucleotides 19a, 19b and 19c forming no duplexes are removed from the sample solution, which in turn is thereafter analyzed with the ion pair chromatograph shown in FIG. 2.

The separation column 7 is adjusted to the melting temperature of the heteroduplexes 23a and 23c with the column oven 9. The feed pump 3 feeds the acetonitrile solution containing triethylamine to the separation column 7 as the mobile phase while adjusting the concentration of acetonitrile by the gradient elution apparatus 1. The sample solution is injected from the injector 5, mixed with triethylamine and introduced into the separation column 7. When the sample solution is mixed with triethylamine, triethylamine is coordinately bonded to phosphoric acid groups of the homoduplexes 21a, 21b, 21c and 23b and the heteroduplexes 23a and 23c contained in the sample solution, to improve hydrophobicity of these portions.

When the sample solution is introduced into the separation column 7 in this state, the heteroduplexes 23a and 23c are dissociated in the separation column 7 in a higher ratio than the homoduplexes 21a, 21b, 21c and 23b since the separation column 7 is adjusted to the melting temperature of the heteroduplexes 23a and 23c. Retention power of the separation column 7 for the labeled oligonucleotides 19a and 19c dissociated from the chain 23 is so reduced that the oligonucleotides 19a and 19c elute in advance of the hybridized labeled oligonucleotides 19a, 19b and 19c.

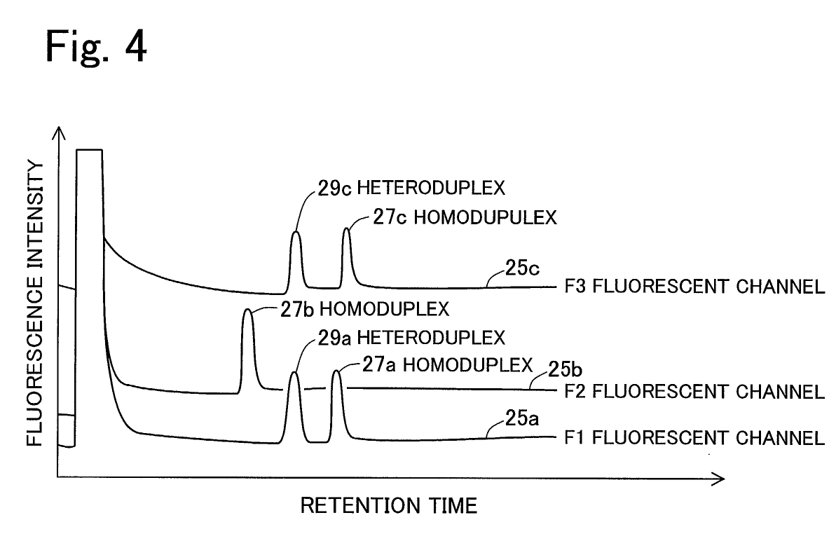

FIG. 4 is a waveform diagram showing a chromatogram in this embodiment. Referring to FIG. 4, the vertical axis shows intensity of fluorescence, and the horizontal axis shows retention time.

Two detected peaks 27a and 29a appear on a detected waveform 25a of an F1 fluorescent channel, a single detected peak 27a appears on a detected waveform 25b of an F2 fluorescent channel, and two detected peaks 27c and 29c appear on a detected waveform 25c of an F3 fluorescent channel. Each of the detected waveforms 25a and 25c has two detected peaks since those of the labeled oligonucleotides 19a and 19c forming the heteroduplexes 23a and 23c have eluted in advance. In other words, the detected peaks 27a, 29a, 27b, 27c and 29c show the presence of the homoduplex 21a (including the non-dissociated heteroduplex 23a), the heteroduplex 23a, the homoduplexes 21b and 23b, the homoduplex 21c (including the non-dissociated heteroduplex 23c) an the heteroduplex 23c respectively.

Thus, it is understood that there has been mutational base sequence in the inspected sites corresponding to the exons 15a and 17a and the exons 15c and 17c of the object of analysis before the PCR amplification.

The present invention is not restricted to the aforementioned embodiment, and the structures of the ion chromatograph and the reversed phase column, the mobile phase and the ion pair reagent are not restricted to those in this embodiment either.

While mutation in the base sequence is inspected as to three inspected sites present in the same DNA fragment in the aforementioned embodiment, the object of analysis in the present invention is not restricted to this but may also be the overall nucleic acid including a plurality of types of inspected sites of base sequence, or that prepared by mixing a plurality of nucleic acid fragments including inspected sites, or a mixture of these.

While the reversed phase column is adjusted to the melting temperature of the heteroduplexes in the aforementioned embodiment, the present invention is not restricted to this and a temperature causing difference in stability between hetero- and homoduplexes may be employed.

Although the present invention has been described and illustrated in detail, it is clearly understood that the same is by way of illustration and example only and is not to be taken by way of limitation as the spirit and scope of the present invention are limited only by the terms of the appended claims.

What is claimed is:

1. A method of detecting mutation in the base sequence of nucleic acid, including:
(A) a bonding step of hybridizing an object of analysis consisting of nucleic acid or a nucleic acid fragment including a plurality of inspected sites to be subjected to inspection of mutation in the base sequence with a plurality of types of oligonucleotides having base sequence complementary to any of the inspected sites having normal base sequence and labeled to be discriminable from each other for forming duplexes; and
(B) a detection step of employing an ion pair chromatograph comprising a reversed phase column serving as a separation column and a detector capable of discriminating and detecting the labeled oligonucleotides and setting the separation column at a temperature causing difference in stability between hetero- and homoduplexes included in the duplexes for analyzing the object of analysis.

2. The mutation detecting method according to claim 1, wherein
the oligonucleotides are labeled with the fluorescent materials.

3. The mutation detecting method according to claim 1, wherein
the separation column is set at the melting temperature of the heteroduplex.

4. The mutation detecting method according to claim 1, observing a chromatogram of labels obtained through the detection step (B) for determining an inspected site corresponding to a label having a single peak as non-mutational while determining an inspected site corresponding to a label having two peaks as mutational.

5. The mutation detecting method according to claim 1, including an amplification step of amplifying the object of analysis in advance of the bonding step (A).

6. The mutation detecting method according to claim 5, wherein
the amplification step is a single PCR step.
